(19) United States
(12) Patent Application Publication
(10) Pub. No.: US 2008/0286388 A1
(43) Pub. Date: Nov. 20, 2008

(54) Pharmaceutical Composition and Non Dependence Coffee Comprising Edible Carboxylic Acid and/or Its Acid Salts and Coffeine

(76) Inventor: Shin-Jen Shiao, Taiwan (CN)

Correspondence Address: Shin-Jen Shiao, 4F-6, No. 98 Jianzhong Road, Hsinchu 300, TW

(21) Appl. No.: 11/576,955

(22) PCT Filed: Oct. 24, 2005
(86) PCT No.: PCT/CN2005/001749

§ 371 Date: Jan. 3, 2008

(30) Foreign Application Priority Data
Oct. 22, 2004 (CN) PCT/CN2004/001200

(51) Int. Cl. A61K 36/258; A61K 36/00; A61K 36/8962; A61P 35/00; A61K 36/18; A61K 36/736

(52) U.S. Cl. 424/728; 424/725; 424/754; 424/773; 424/765

(57) ABSTRACT

Pharmaceutical composition and food comprising edible carboxylic acid and/or its acidic salt and optionally caffeine, crude drug and pharmaceutical acceptable carrier are disclosed. The invention also discloses the use and method of composition or food. The composition or food of the present invention can prevent, treat or relief allergy, ache, cold, viral infection, thrombus or clotting, inflammation, cancer, intoxication, memory decay, caffeine dependence. The invention also relates to feedstuff for animals.

### CROSSE LINKING REFERENCE OF THE INVENTION

This invention refers to the contents of the application of PCT/CN2004/000402 filed Apr. 26, 2004.

### FIELD OF INVENTION

This invention relates to pharmaceutical composition to prevent, treat and alleviate allergic diseases, ache, infection, cold, thrombus or clotting, inflammation, cancer, viral infection, intoxication, memory decay, caffeine dependence by decreasing body fluid pH, and their health care foods. This invention also relates to non-addicted coffees, their usages and preparation methods thereof. And also relates to feedstuff for animals.

### BACK GROUND OF THE INVENTION

There are four types of hypersensitivity reactions: Type I is immediate type mediated by IgE causing diseases such as anaphylaxis, dermatitis, asthma, Parkinsonism, hay fever, and food allergy. Type II is cytotoxic type mediated by antibodies of IgG and IgM causing diseases such as haemolytic disease of the newborn, autoimmune haemolytic anemia, acute rheumatic fever, nephritis, drug allergy and hepatitis. Type III is immune complex type mediated by hypersensitivity causing diseases such as lupus nephritis, Arthus reaction, rheumatoid arthritis, vasculitis, and serum sickness. Type IV is delay type hypersensitivity mediated by T cell hypersensitivity which causes diseases such as Type I hypersensitivity, erythema, and multiple sclerosis.

Immunodeficiency is divided into inherited immunodeficiency and acquired immunodeficiency. The latter is caused by human immunodeficiency virus, and the former causes diseases such as respiratory infections, herpes simplex virus, chronic lung pneumonia, influenza, and skin inflammation. However, scientists hope to develop vaccines against these diseases, not effective vaccine is found yet.

There are three groups of drug treating immunological disorders: first, anti-inflammation drugs of the corticosteroid family, such as prednisone and antihistamine; second, cytotoxic drugs, such as azathioprine and cyclophosphamide; and third, fungal and bacterial derivates, such as cyclosporine-A and rapamycin, which inhibit signaling events within T lymphocytes.

These drugs have wide action in inhibiting immune system as well as harmful ones. The beneficial effects of corticosteroids are anti-inflammation. However, there are also many side effects, including fluid retention, gain of weight, diabetes, bone mineral loss, and thinning of skin. They are caused by the results of using corticosteroids which reduces the functions of hormone and also reduces the immune functions too.

The cytotoxic drug suppresses immune by killing cells. That has serious side effects, including decreasing immune function, anemia, damage to intestinal epithelium, hair loss, and fetal death or injury.

The drugs of fungal and bacterial derivatives are toxic to kidney and other organs. Besides, it is expensive to ingest for a long period of treatment.

Histamine is a kind of harmful secretions in allergic reaction. That is a potent mediator in numerous biological reactions. Following the stimulation of mast cells and basophils by antigens, histamine and other compounds are released explosively into the surrounding tissues and body fluids. On releasing, histamine functions a potent mediator of numerous physiological, and causes pathophysiological processes in all organs and tissues. That immediately effects a dilation of the blood vessels, so that fluid escapes into the surrounding tissues. This reaction may result in a general depletion of vascular fluid causing a condition known as histamine poisoning or histamine shock.

Antihistamines are used primarily to control symptoms of allergic diseases such as hay fever. Chemically, antihistamines comprise several types. Each antihistamine neither cures all kinds of syndromes nor is good for any person. Side effects of these drugs include drowsiness, loss of concentration, and dizziness.

The traditional antihistamines are compounds of amine. As you know, amines have properties of high alkaline, toxic to body, damage to the stomach, and low solubility in water. That the amine does not suitable to be a drug. For improving, chemists applied acids, including organic acid and inorganic acid to react the amine compound to form a salt. There are many acids including inorganic acid: such as hydrogen chloride; and organic acids, such as maleic acid, citric acid, malic acid, tannic acid and succinic acid; are used.

In a diphenhydramine system, for example, the diphenhydramine is reacted with hydrogen chloride to form diphenhydramine hydrochloride; and in a chlorpheniramine system, the chlorpheniramine is reacted with hydrogen chloride to form chlorpheniramine hydrogen chloride. The other compounds such as chlorpheniramine maleate, phenyltroxamine citrate, diphenhydramine tannate, diphenhydramine salicylate, and chlorpheniramine malate are products of reaction with organic acids of maleic acid, citric acid, tannic acid, salicylic acid and malic acid, respectively. The role of acid, such as hydrogen chloride, maleic acid, citric acid, malic acid, salicylic acid, or tannic acid, is used a modifier. They neutralize the alkalinity of amines, lower the amine toxicity for patients, and increase the solubilities thereof. This is the origin of traditional antihistamines which are used widely to treat allergic diseases now.

Food poisoning and insect bit are two kinds of poisoning in daily life, normally. The former is caused by eating foods containing disease bacteria or toxin; and the later is caused by venom of insect bite. This toxicity could cause serious allergic reaction, and may be considered a kind of allergic diseases. The traditional treatment use anti-toxin and modified toxins for bacterial toxins (such as Diphtheria, tetanus toxin), and use antivenoms for insect venoms (such as black widow, snake). They are produced by vaccinating repeatedly in other animal species. Infusion a large amount of antibodies into the body will induce hypersensitivity. The disadvantage of this method is that must test in advance to make sure that the patient has not allergy history.

As a result of study, the applicant found that if supply protons to react all the base amino groups and histamine receptors in humoral, there is not any hypersensitivity reaction will happen. This is the basic principle invented of the invention. On top of that, this principle is also applied to reactions of physiology and pathology that take place between the receptors of cells and substrates such as protein containing substrate (normal antigens or pathogens), toxins, and drugs. All of these substrates can react with receptors of proteins. Therefore, to decrease body fluid pH by carboxylic acid compounds, supplying protons, could gain the goals of preventing, treating or alleviating diseases such as allergy diseases, ache, cold, viral infection, thrombus or blood clotting, inflammation, cancer, intoxication, and caffeine dependence.

Because almost all invaded pathogens, including toxin, contains active residues of protein such as hydrophobic nueclphilic groups, e.g. amino acid residues, can react with histamine receptors. Many drugs also contain such amino acid residues, and react with histamine receptors of cells and causing reactions. The functions of the drug composition of this invention inhibit hypersensitivity reactions by neutralizing nueclphilic groups of invaded pathogens or blocking the histamine receptors. Applying this function improve the side effects of drugs. Taking Paclitaxel, for example, though is claimed to treat many cancers, has side effects such as lowering leucocytes, fever, vomit, diarrhea, inflammation of mouth corner and dropsy. These problems can be improved by combining the application of carboxylic acids. Besides, food allergy also is caused by active proteins contained in food. That can also be improved in combination use or pretreatment with carboxylic acid to inhibit or neutralize the active sites of protein.

As for the infection of cold, applying protons and anions dissociated from carboxylic acid block the active sites of virus and receptors of cells, respectively. That separate viruses from close to cell membrane, inhibiting viruses approach to cell receptors. Taking HIV (human immunodeficiency virus) for example, to approach cell receptors must be in a neutral condition. If not, the virus would not come near to the target cell receptor, and never get into cell for infection. The functions of this invention itself decreasing pH is for this purpose. That function supplies protons combining nucleophilic amino residues and inhibiting the contact of virus and cell membrane, that preventing further binding and fusion reactions with cell.

Abilities of learning and memory are very important for human being. The most anguish matter for student may be the low efficacy of study caused by poor ability of memory. Problems such as losing memory with age and even the worst Alzheimer's disease are all caused by degenerative disease of brain nerves. The man evolution is a story of developments of learning and memory, because the survival of the fittest in competitive society is determined by the abilities of learning and memory. Thus, an effective method to increase abilities of learning and memory would be a great contribution to human beings. It has been studied that the muscarinic receptors of brain nerves play a vital role in learning and memory, and their agonists also are neurotransmitters of central nervous system. The neurotransmitters, acethylcholine, transmit signals at synapse, but would lose their functions when the amount of acetylcholine is scant. There is a way to increase the quantity of acethylcholine by inhibit the activity of acethylcholinase. To increase amount of acethylcholine which combine receptors of muscarine and choline, may increase the signals transmitting of nervous system, and improve the abilities of learning and memory. The acethylcholinase can be hydrolyzed by acethylcholinesterase at a rate of 104 per second, so that to reduce the hydrolysis strength of acethylcholinesterase is very important to keep the level of acethylcholine. Many drugs have been developed but not successes for the reasons of toxic and side effects. The applicant found this invention just meets the purpose and without any side effect at all. The mechanism, not limited, may be due to the active site of acethylcholinesterase is the negative charges of glutamate and tryptophan. This negative charge abstracts the ammonium group of acethylcholine, and then hydrolysis takes place at another end. If the negative charges are neutralized by protons, the acethylcholinesterases would lose their functions. To decrease body fluid pH by application of this invention supplying protons can neutralize the negative charges of acethylcholinesterase. By that, a lot of neurotransmitters, acetylcholines, can function and increase the efficacies of learning and memory, and let human being smarter. This reaction also can be applying to improve diseases such as Alzheimer's disease. (K. P. Minneman et al., Bordy's Human Pharmacology 4th, Elesvier Mosby, 2005, China.) The invention use carboxylic acid to decrease the pH and inhibit the hypersensitivity, and improve the efficacies of learning, memory and works greatly.

Caffeine is a central nerves system stimulant. That will be addicted and causing caffeine allergy when ingesting large amount or using frequently. The well being, alert and stay awake are caused by the receptors of adenosine and dopamine being blocked by ingested caffeine. Larger amount and more frequent doses are needed for the same effect at next time, and symptoms can develop if we do not get over our “fix”. Eventually, we need the drug to function; without it, fatigue and drowsiness occur. Caffeine dependence is one of health problems which are treated to date with histamines or aspirin, and causing side effects. Using this invention the caffeine dependence can be improved without side effects.

Another bad effect of coffee is that the drinker can not assimilate minerals such as calcium, iron and magnesium from food, which makes the body short of these minerals and causing diseases. Besides the refresh effect of coffee, another bad point which is needed to dissolute is no nutrition.

There is a drug containing equal mole of caffeine and citrate called caffeine citrate, Cafcit, which is used to treat apnea of premature infants. Cafcit has caffeine toxic and other adverse events such as nucleolar hyperactivity, muscle tremors, jitteriness, and tachycardia. The citric acid component of Cafcit is an acid which is used to neutralize alkaloids as the same in traditional histamines described previously. The amount of organic acids used in present invention is greater than caffeine, normally a few times greater than caffeine. Therefore, the composition, functions and purpose of the invention are completely different from Cafcit.

The study results show that the problem of coffee drinkers can not assimilate minerals such as calcium, iron and magnesium from food can be improved by containing organic acids in coffee which makes the citrates being adsorbed easily by intestines. Moreover, for the nutrient problem of coffee drinks the inventor also composite organic acids, herbs and nutrients to coffee drinks. That each component function of drinks plays synergism: caffeine stimulating the reaction rate of carboxylic acid; improving the taste of acid, perfume and well being. In the other hand, the organic acids improve the problems of caffeine dependence and toxin; and dissolve severe problem of disturbance of assimilation of minerals such as calcium, iron and magnesium from food. This synergism effects of one another make the coffee drinks becomes a good one. That is the contribution of this invention.

Drugs of inflammation and analgesic are divided into steroid and non-steroid: the latter also divided into anesthetic and non-anesthetic. The anesthetic analgesics such as morphine and codeine have problems of physical dependence, oliguria, low body temperature, constipation, respiration inhibition, and itch. Non-steroid anti-inflammatory and ache drugs such as aspirin and acetaminophen have side effects. That made the inventor researching to improve these problems, and found drugs for inflammation and ache without side effects.

This invention also can treat cold effectively.

Sodium citrate can react with calcium ions and is used as anticoagulant in transfusion of blood. Thrombus plays a vital role in inducing cardiovascular disease. Drugs of this invention not only can fix calcium component, but also inhibit free radicals and the activities of phosphalipase and cyclooxygenase; the cascade formation of prostaglandin; and release of thromboxane A2. Thus, the formations of embolus and thrombus, cardiovascular residuals of cholesterol and triglyceride are inhibited, and then can eliminate the possible formation of thrombus, finally. In addition, drugs of this invention can be used as anticoagulant in transfusion blood and blood dialysis instead of injection of conventional anticoagulant.

A method of treating prostate cancer for males by administration of tartrate ions from a tartrate derivative, the tartrate ions in the bloodstream bind to and inhibit the activity of prostatic acid phosphatase was disclosed by Lebioda et al. in U.S. Pat. No. 5,763,490. This disclosure uses cations of tartrate binding to acidic prostatic acid phosphatase and decreasing it, when its pH is not decrease but increase. That is completely different from the basic principle of present invention which uses protons of tartaric acid to decrease the body fluid pH. A process for the production of fruit polyphenols from unripe rosaceous fruits is disclosed by Tanabe et al. in JP3-254340 and JP7-285876. They claimed the polyphenol extract, the main effective component, which containing a minor component of organic acids, has physiological activities such as antiviral activity, anti-allergic activity, antioxidative activity and antimutagenic activity. Siraishi et al. disclosed an antimutagenic agent comprises at least one kind of an organic acid in JP2003-104880. In addition to these organic acids, the inventor found that the addition of another component such as garlic and caffeine can enhance the antimutagenic activity of organic acid. Paul and John disclosed compositions for prevention and treatment of cold and influenza-like systems and their methods of use in WO 012855A and JP-T-2003512335T. That disclosure claimed a composition using pyrrolidone carboxylic acid as main effective component and then adding organic acid, neutralizing the solution to pH 3.5-5.5 with sodium hydroxide consequently, and is used to treat respiratory tract viral infections. This drug can only treat respiratory but not whole body. Besides there are defects as follows: First, the main ingredient of pyrrolidone carboxylic acid is toxic. Second, for the toxic the drug can not oral administrate. Third, after organic acids are added to the effective component of pyrrolidone carboxylic acid, the solution is neutralized with strong alkaline sodium hydroxide to pH 3.5-5.5 to form a nasal drug. The treatment of disease is not caused by lowering the pH value of humoral fluid, referring to examples of I-V of this specification.

Sie. I. disclosed using 0.1-2% (v/v) of acetic acid solution to treat respiratory disease, but do not concern to oral administration in CN 1564325. Kinyuan et al. reported that tartaric acid is used to treat convulsions, analgesia, rheumatism, gout, scabies and snake toxin (Pharmacology, February 1983, vol. 18, No. 2, pp 36-38.) In addition to the tartaric acid, the present inventor found that the addition of other components such as garlic and caffeine can enhance its activity. Kurtasova disclosed a method for treating atopic bronchial asthma in RU 2236849 that one should introduce causal-valuable allergen and, additionally, succinic acid properly not more than 0.05 g twice daily after meals can increase the immunity of cells and decrease the allergic inflammation. In addition to this succinic acid, the present inventor found that other components such as garlic and caffeine can enhance its effective.

Cheng gang disclosed, CN 1112956, a beer herbal additive in which contains caffeine and citric acid, and in order to decrease the acidity adding alkaline sodium bicarbonate (B group of ingredient) is claimed to lasting the bubbles and improving its taste. The purposes and methods of that disclosure are basically different completely from present invention that decreasing body fluid pH to prevent or alleviate diseases. Cheng Inje et al. disclosed a composition for dispel the effects of alcohol in CN 1080864 in which claimed most of composition being herb such as SAGI fruit, fresh orange, licorice and ginseng. The purpose of that disclosure is different from present invention that decreasing body fluid pH to prevent or alleviate diseases and treatment of caffeine addiction.

R. K. Joshi et al. disclosed, CN 1316901 A and U.S. Pat. No. 6,359,003, drugs using fumaric acid and/or its esters derivatives for transplantation; K. Kuroda et al. disclosed m, CN 1325302A and U.S. Pat. No. 6,509,376B1, drugs using dialkyl fumarates for treatments of transplantation, Ehrlich ascites tumor, toxic effects of mitomycin C and aflatoxin, and psoriasis. All these fumaric acids esters derivatives have toxic side effects. In addition to the fumaric acid, the present inventor found that the addition of another components such as garlic and caffeine can enhance its activity, and without toxic effects of fumaric acid derivatives.

The situations of treatment and defects of drugs up to date for diseases of hypersensitivity reaction, analgesic, cold, infection, thrombus or clotting, inflammation, cancer, viral infection, intoxication, memory decay, caffeine dependence described previously, the inventor found that using edible nature carboxylic acids to decrease body fluid pH can increase the production of complements, enhancing the activities of cells such as marcophage, CD4 T cell and B cells in physiological and treatment. In combing decreasing body fluid pH by edible nature carboxylic acids, herb and applying partial function of coffee, the inventor made present invention that not only improve the defects of coffee but also further increase the effects of treatment and alleviation.

It is believed by most people to date that keeping the body fluid in alkaline is important to health, and will be sick at acidic condition. They must be certainly surprised that diseases are prevented and alleviated by decreasing body fluid pH claimed by present invention. According to the invention, the drug effects the pH at the peak after tow hours of administration, and return to normally condition after four hours, when there are metabolized completely without any residuals. Accordingly, it is an object of the present invention to provide pharmaceutical compositions prevent, treat and alleviate supersensitive reaction diseases.

It is another object of the present invention to provide use of pharmaceutical compositions which prevent, treat and alleviate supersensitive reaction diseases.

It is therefore a general object of this invention to provide pharmaceutical compositions which prevent, treat and alleviate caffeine addiction.

It is still more specific object of this invention to provide non-addiction coffee drinks and preparation methods thereof.

It is the primary object of this invention to provide analgesic compositions which prevent, treat and alleviate ache.

It is the principle object of this invention to provide pharmaceutical compositions which prevent, treat and alleviate cold or viral infection.

It is the most important of this invention to provide pharmaceutical compositions of anti-inflammation.

It is among the objects of the subject invention to provide anti-coagulant composition which treat the problems of thrombus and clotting.

A further object of the present invention is the provision of pharmaceutical compositions which prevent, treat or alleviate cancer.

Still a further object of the invention is the provision of pharmaceutical compositions which prevent, treat or alleviate Alzheimer's disease, and increase the ability of learning memory.

Another important object of the present invention is to provide the usages which decrease body fluid pH by nature edible carboxylic acids and/or their acidic salts, to prevent, treat or alleviate hypersensitivity reaction diseases, ache, cold, viral infections, thrombus or clotting in transfusion of blood or hemodialysis, inflammation, cancer, toxication, memory decay, or caffeine dependence.

An additional object of this invention includes the provision of feeds which is to prevent, treat or alleviate animal hypersensitivity reaction decreases and viral infections. Other and further objects, features and advantages of the invention will appear more fully from the following description.

### Invention Context

Surprisingly, the invention found that edible organic acid, especially, carboxylic acids and their acidic salts such as: succinic acid, fumaric acid, maleic acid, α-hydroxyl acids, malic acid, tartaric acid, citric acid, lactic acid, oxalactic acid, α-hydroxy octanoic acid, gluconolactone, glycollic acid, keto-glutaric acid, aconitic acid; their acidic salts of sodium or potassium; acetic acid, propionic acid; and their compounds; can be used to decrease body fluid pH. These compounds are effective to prevent, treat or alleviate hypersensitivity reaction diseases, ache, cold, viral infections, thrombus or clotting in transfusion of blood or hemodialysis, inflammation, cancer, toxication, memory decay, or caffeine dependence. In addition to that, the addition of other components such as caffeine can raise their activity effectively. Monoacidic salts can be formed in the condition of mixing solutions of acid and its salts. It also takes place in body fluid after ingestion. That the compounds mixed with acid and it salt are also belonging this invention. As for the use of coffee, caffeine, coffee powder, extract of coffee or extract of caffeine containing vegetables are also included in this invention.

The applicant invented that edible acid and/or its acidic salt as active agent for the treatment and alleviation of allergic diseases by decreasing pH are acids, such as fumaric acid, succinic acid, α-hydroxyl acids, malic acid, tartaric acid, citric acid, lactic acid, oxalactic acid, α-hydroxy octanoic acid, gluconolactone, glycolic acid, their acidic salts of sodium and potassium, acetic acid, porpoinic acid, and their compounds; show wonderful effective in treating allergic disease.

There are oral and non-oral usages of drugs of present invention used in prevention, treatment and alleviation diseases. The normal therapeutic dose is about 0.1˜300 mg/kg/day. The dose could be much more than that according to necessary, in which the caffeine content in each dose preferable is must less than 200 mg, more preferable is less than 50 mg. They can be prepared in any forms of drug by the known pharmaceutics, and even combining with other active components. It is a common knowledge that herbs active components must be extracted when use in preparing injections.

Drugs used to prevent, treat and alleviate of caffeine dependence, the caffeine content must be reducing to nothing gradually.

For non-caffeine dependence coffee drinks, there are many methods for preparing the drugs including: to make in a form of powder with other components, adding into solution; even being packed organic acids and/or another components of nutrients separately, and adding after coffee is prepared; and to ingest before or after drinking coffee.

Routes of drug administration of present invention may be by parenteral method, including subcutaneous, intramuscular, intravenous, intradermal, transdermal, and by external pathways. Non-oral external drugs made by traditional method including plasters, tinctures and skin plasters. The liquid solvent includes water, alcohol, glycerin, and other glycols. The other compatible active agents can be contained in the present invention. The oral drug of present invention can be in the forms of capsule, tablet, flake, powder, pile, lozenges, syrup, solution and suspension.

The said organic carboxylic acids and/or their acidic salts and caffeine of present invention also can be preparing non-addition coffee.

The said decreasing body fluid pH by organic carboxylic acids and/or their acidic salts and caffeine of present invention has pain killing effect that can be used as analgesics.

The compositions of oral drinks, food or coffee drinks of present invention can contain any other components selected from the group comprised such as: binder, inert; dilution; agent; thickener; softener; dispersion agent; emulsifier; preservative; lubricant; enzyme; sweetener; perfumer; pigment; herbs such as: Gambier, areca and its products, garlic, leek, chive, shallot, ramson, scallion, zinger, tang-kuei, licorice, astragali radix, almonds, ginseng, atractylodis rhizoma, pinelliae tuber, angelica sinensis radix, hoelen, asini gelatinum, dried tangerine, asparagi radix, rehmanniae radix et rhizoma, perillae fructus, perillae caulis, anemarrhenae rhizoma, albae sinapis semen, mori radicis cortex, zingiberis siccatulm rhizoama, yam, carroten, lily bulbous, sesame, ginseng, in powder or extracts; processed fruits, other nutrition such as mineral, vitamin, powder milk, peanut product; vegetable seed oil, cooked foods, amino acids; and their compounds.

One formula of the invention which decreases body fluid pH to prevent, treat or alleviate hypersensitivity reaction diseases, analgesic, viral infection, cold, thrombus or clotting in transfusion blood or hemodialysis, inflammation, cancer, intoxication, memory decay, caffeine dependence is 4˜100 wt %, preferable is 4˜94 wt %, more preferable is 10˜90 wt %, the best is 15˜85 wt % of edible organic carboxylic acids and/or their acidic salts; 0.1˜6 wt %, preferable is 0.1˜5 wt %, more preferable is 0.5˜4%, the best is 1˜3 wt % of caffeine as active component; 0˜80 wt % of herbs; and 0˜90 wt % of pharmaceutical acceptable carriers. The amount of edible organic carboxylic acids and/or their acidic salts is greater than that of caffeine, the best is greater more than three times, and the amount of caffeine in each dose preferable is less than 200 mg, more preferable is less than 50 mg.

One further formula of the invention which decreases body fluid pH value to prevent, treat or alleviate hypersensitivity reaction diseases, aache, vial infection, cold, thrombus or clotting in transfusion blood or hemodialysis, inflammation, cancer, intoxication, memory decay, caffeine dependence is 4˜100 wt %, preferable is 5˜95 wt %, more preferable is 10˜90 wt %, the best is 15˜85 wt % of edible organic carboxylic acids and/or their acidic salts as active component; 0˜6 wt %, preferable is 0.1˜5 wt %, more preferable is 0.5˜4%, the best is 1˜3 wt % of caffeine; 1˜80 wt % or 0% of herbs; and 0˜80 wt % or 0˜96% of pharmaceutical acceptable carriers. The amount of edible organic carboxylic acids and/or their acidic salts is greater than that of caffeine, the best is greater more than three times, and the amount of caffeine in each dose preferable is less than 200 mg, more preferable is less than 50 mg.

In addition to the to prevent, treat or alleviate said diseases by simple decreasing body fluid pH, the inventor also found that combine edible organic carboxylic acids and/or their acidic salts, stamina garlic and caffeine would promote the drugs effective much more. One formula of that is 14˜89.9% of edible organic carboxylic acids and/or their acidic salts, 10%˜80% of garlic, 0.1%˜6% of caffeine, and 0˜80% of pharmaceutical acceptable components. These compositions are not limited because the frequency and dose are depending on syndromes and speed of treatment. Moreover, their components are food without toxicity, so that is nothing to do with limitation.

This invention also found that the side effects of drug of Paclitaxel can be alleviate or improved by edible organic acids and/or their acidic salts. The method is that treating the injection drug with edible organic carboxylic acids and/or their acidic salts; injecting the composition of two drugs; treating with edible organic carboxylic acids and/or their acidic salts after injection of Paclitaxel; or instead of other drugs such as steroids and antihistamines using directly with edible organic carboxylic acids and/or their acidic salts; when the amount of edible organic carboxylic acids and/or their acidic salts is greater than Paclitaxel. Thus, the present invention provides a pharmaceutical composition that prevents, treats or alleviates the side effects of Paclitaxel in which includes Paclitaxel, edible organic carboxylic acids and/or their acidic salts, and other active components. In the preparation of Paclitaxel injection composites with fine powdered edible organic carboxylic acids and/or their acidic salts in a ratio of one to five times. To use two drugs separately has a more selectivity, and can adjust the dose depending the patient needs.

The invention also is applied to animal drugs to prevent, treat and alleviate relative diseases of spondyle animals like what human beings do. It is better to make the effective oral drugs in the form of microencapsules or dispersing the powder in lime stone powder that mixes with feedstuff finally. Therefore, the invention provides a animal feedstuff additive in which contains 4˜94%, preferable is 5˜90%, more preferable is 10˜90%, and the best is 15˜85% of edible organic carboxylic acids and/or their acidic salts; 0.1˜6%, preferable is 0.1˜5%, more preferable is 0.5˜4%, and the best is 1˜3% of caffeine as active components; 0˜80% of herbs; and 0˜96% of pharmaceutical acceptable carrier. This invention also provides a feedstuff in which contains the said animal feedstuff additive and regular ones. This feedstuff also can be prepared by normal methods such as compounding, blending and coating.

This invention uses edible acids. Therefore, the edible organic carboxylic acids and/or their acidic salts can be used as additive in health care food. Their carriers are food acceptable compounds including raw materials that are processed from acid containing fruits, such as orange, acid orange, lemon, plum, grape fruit, star fruit, mulberry, strawberry, and pineapple. The types of health care food are normal oral food such as drinks, candy, biscuits, capsule, tablets, flake, granular, powder, pile, syrup, solution, and suspension. In which contains 0.1˜10% of edible organic carboxylic acids and/or their sodium or potassium acidic salts; 0˜6%, preferable is 0.1˜5%, more preferable is 0.5˜4%, and the best is 1˜3% of caffeine; 0˜80% of herbs; and 0˜90% of pharmaceutical acceptable carrier. The amount of edible organic carboxylic acids and/or their acidic salts is greater than that of caffeine, the best is greater more than three times, and the amount of caffeine in each dose is preferable less than 200 mg, more preferable is less than 50 mg.

The food hypersensitivity risks can be improved by edible organic carboxylic acids and/or their acidic salts. The method is that the food is pretreated with edible organic carboxylic acids and/or their sodium or potassium acidic salts in which the amount of said acids are 0.1˜10%. The invention also relates to the health care food itself.

All the amounts said in this invention are in dried basis.

The pharmaceutical composition of this invention can be produced by the well known arts in relative field and many published literatures can be referred, including such as Remington's Pharmaceutical Science.

In this invention, the effective testing are carried out with mice and human bodies, these are used to describe the invention but not limited the scopes. The “individual” means any spondyle animal, including poultry and mammal such as pig, dog, cat, horse, cow, monkey, sheep, goat, rabbit, gorilla, human, chicken, duck, goose, bird, and the best is human.

### CONCRETE EXAMPLE

This invention will be understood more readily with reference to the following examples. These examples, however, are intended to illustrate the invention and are not to limit the scope of the invention. In examples, some examples of international patent application PCT/CN2004/000402 (experimental results of non-caffeine addition) are used for comparison.

### Example 1-32

### Anti-Allergy Reaction

This is a comparative testing for drug depressing effect on the amount of leaching histamines when is treated with 48/80 (Sigma, St. MO, USA) compound.

(1). Preparation of Leaching Cell Solution from Mouse Body.

A mouse is killed and bloodletting. Then 10 ml of Locke's solution containing 0.1% bovine serum protein is injected into its abdominal cavity. After abdominal cavity being light massaged, the cavity is cut and the Locke's solution is removed. Cavity is washed with another 5 ml of Locke's solution, and this washed solution is added to the latter one. This combined solution is centrifuged at 600 rpm for 5 minutes. The sediments are washed with 5 ml of cool Locke's solution. Adding 3 ml of cold Locke's solution to the washed sediments, then a leached cell solution of abdominal cavity is obtained. (The composition of Locke's solution is: NaCL 9.1%, KCL 0.2%, CaCL2 0.15%, glucose 1.0%, in w/v, and the rest distillated water.)

(2). Drug Inhibiting Effect on the Amount of Leaching Histamines when Treated with 48/80 Compound.

Each testing compound (edible acid/acidic salt and its 5% of coffee hot water extract) listed in the table 1 is dissolved in a Ringer's solution containing 1% NaHCO3, and then diluted with Locke's solution to the indicated concentration of 100 μg/ml. 1.0 ml of each of those solutions in last term is mixed with 0.3 ml of mouse's leaching cell solution and 0.5 ml of Locke's solution. This mixture is cultivated at 37° C. for 5 minutes. Then adding 0.2 ml of Locke's solution of 48/80 compound (1 mg/100 ml) and cultivated at 37° C. for 10 minutes. Then the reaction is stopped by cooling, and centrifuged at 2,500 rpm for 10 minutes. 1.7 ml of decanted solution and 0.3 ml of sediments are obtained.

0.1 ml of water and 0.2 ml of 100% trichloroacetic acid are added to the decanted solution. 1.5 ml of Locke's solution and 0.2 ml of 100% trichloroacetic acid are added to the sediments washed solution. They are cultivated at room temperature for 30 minutes. After cultivation, the mixtures are centrifuged at 3,000 rpm for 15 minutes, respectively. 0.35 ml of each of the former two solutions is sampled. In each sample, 1.65 ml of water, 0.4 ml of 1N NaOH and 0.1 ml of 0.5% OPT (o-phthalic aldehyde) in methanol are added and cultivated at room temperature for 4 minutes. The reaction is stopped by adding 0.2 ml of 2M citric acid. And finally, determine the amount of released histamines in the tested solution by fluorescence method. By the analysis, results of the inhibiting rate of histamines could be calculated.

Locke's solution is instead of each drug in control group, and instead of both drug and 48/80 compound solution in blank group.

The experimental dose of 100 μg/ml is used in order to comparison with the effective of traditional drug. The advanced effects are improved by using different doses the drugs of succinic acid and sodium glycyrrhizinate, and testing the concentration for inhibiting rate of 50% (IC50 value).

The histamine releasing rate (A) can be calculated by following equation. Where (Hs) is the total amount of histamine in the decanted solution, and (Hr) is the total amount of histamine in the sediment. (A)=[(Hs)/{(Hs)+(Hr)}]×100%. Then the inhibiting rate is:

=100−[(A−A in blank group)/(A−A in control group)]×100%.

The calculated results are shown as following table 2.

TABLE 1
Drug depressing effect.

| | | Histamine | inhibiting rate (%) | Inhibiting rate (%) |
|---|---|---|---|---|
| Testing | Testing drug | releasing | (adding 5% of coffee | (non adding coffee |
| No. | 100(μg/ml) | rate (%) | extract) | extract) |
| | Control group | 90.8 | — | — |
| | Blank group | 90.5 | — | — |
| (1) | Trisodium glycyrrhizinate | 51.5 | 48.3 | 30.9 |
| (2) | Diphenhydramine hydrochloride | 49.8 | 50.4 | 32.1 |
| (3) | Diphenhydramine citrate | 45.6 | 55.5 | 37.5 |
| (4) | Succinic acid | 9.4 | 100 | 100 |
| (5) | Citric acid | 9.4 | 100 | 100 |
| (6) | Lactic acid | 9.2 | 100 | 100 |
| (7) | Malic acid | 9.1 | 100 | 100 |
| (8) | Tartaric acid | 9.4 | 100 | 100 |
| (9) | Fumaric acid | 9.3 | 100 | 100 |
| (10) | α-hydroxy ethanoic acid | 9.4 | 100 | 100 |
| (11) | α-hydroxy octanoic acid | 9.2 | 100 | 100 |
| (12) | gluconolactone | 9.3 | 100 | 100 |
| (13) | acetic acid | 9.3 | 100 | 100 |
| (14) | propionic acid | 9.3 | 100 | 100 |
| (15) | coffee extract | 55.0 | 44.0 | — |
| (16) | sodium dihydrogen citrate | 9.4 | 100 | 100 |
| (17) | disodium hydrogen citrate | 50.6 | 49.4 | 39.1 |
| (18) | keto-glutaric acid | 9.4 | 100 | 100 |
| (19) | aconitic acid, | 9.3 | 40.0 | 100 |
| (20) | oxalacetic acid | 9.4 | 100 | 100 |
| (21) | potassium dihydrogen citrate | 9.4 | 100 | 100 |
| (22) | dipotassium hydrogen citrate | 51.2 | 48.7 | 40.0 |
| (23) | sodium hydrogen succinate | 9.4 | 100 | 100 |
| (24) | potassium hydrogen succinate | 9.4 | 100 | 100 |
| (25) | sodium hydrogen tartrate | 9.4 | 100 | 100 |
| (26) | potassium hydrogen tartrate | 9.3 | 100 | 100 |
| (27) | sodium hydrogen malate | 9.4 | 100 | 100 |
| (28) | potassium hydrogen malate | 9.2 | 100 | 100 |
| (29) | sodium hydrogen maleate | 9.3 | 100 | 100 |
| (30) | potassium hydrogen maleate | 9.3 | 100 | 100 |
| (31) | sodium hydrogen fumate | 9.4 | 100 | 100 |
| (32) | potassium hydrogen fumate | 9.4 | 100 | 100 |

Trisodium glycyrrhizinate and diphenhydramine hydrochloride are traditional antihistamines as shown in Table 1. It is quite obvious that the results of drugs of present invention show completely affective while the traditional drugs are incompletely. For the purpose of showing up the effect of traditional Trisodium glycyrrhizinate drug to be in experimental window, the inhibiting rates of many drugs are 100% because of high doses were used.

TABLE 2
Relationship of dose and inhibiting rate

| | Trisodium glycyrrhizinate | | Succinic acid (adding 5% | | Succinic acid (non-adding | |
|---|---|---|---|---|---|---|
| | (adding 5% of coffee extract) | | of coffee extract) | | coffee extract) | |
| | Histamine | | Histamine | | Histamine | |
| Dose | releasing | Inhibiting | releasing | Inhibiting | releasing | Inhibiting |
| (μg/ml) | rate (%) | rate (%) | rate (%) | rate (%) | rate (%) | rate (%) |
| Control | 90.8 | — | 90.8 | — | 90.8 | — |
| group | 90.1 | 0.8 | 70.5 | 24.9 | 76.2 | 17.9 |
| 2 | 89.5 | 1.6 | 55.3 | 43.6 | 60.1 | 37.7 |
| 5 | 88.1 | 3.3 | 19.3 | 87.9 | 22.5 | 84.0 |
| 10 | 76.3 | 17.8 | 9.1 | 100 | 9.1 | 100 |
| 50 | 65.5 | 31.1 | 9.3 | 100 | 9.3 | 100 |
| 100 | 32.7 | 70.5 | 9.4 | 100 | 9.4 | 100 |
| 300 | 9.2 | 100 | 9.4 | 100 | 9.4 | 100 |
| 1000 | 9.5 | — | 9.5 | — | 9.5 | — |
| blank | | | | | | |

The inhibiting effect value of concentration of 50% can be calculated by figure. The IC50 values of Trisodium glycyrrhizinate (adding 5% of coffee extract), Succinic acid (adding 5% of coffee extract) and Succinic acid (non-adding coffee extract) are 200 μg/ml 5.8 μg/m, and 7 μg/ml respectively. That shows the high effect of succinic acid, and also shows the enhancing effect of addition of caffeine.

The inhibiting effect of histamine can also inhibit the production of compounds inducing by histamine such as 12-HETE, LT, PGX, PGI2, TXA2, PGA2 and PGE2. Histamine is the main cause of inducing the reactions of inflammation, heat, red, analgesic and thrombus.

### Example 33-38 Comparison Testing of Anti-Delayed Type Allergy Reaction

The weights of testing mice are ranging from 20 g to 30 g. They are coated with 0.1 ml of oxazolone alcohol solution (0.5 w/v %) on the hair cleaned part of abdomen. After five days, each of the listed drugs is dissolved in (0.5 w/v %) oxazolone acetone solution in which the drug is 2.0 w/v % with/or without another 0.1 w/v % of caffeine, and 10 μl each of the solutions is token by micro pipette to coat on both sides of the right ear. After 24 hr, the mouse is killed by ether and punched a circle area of a diameter of 5.5 mm on both right and left ears in corresponding part by a puncher machine (portions of drug coated and the blank). The punched portions are weighed and the inflammation rates calculated. The control group are coated only with the oxazolone acetone solution (0.5 w/v %). The inflammation inhibiting rates of each drug are calculated by following equation:

Inflammation inhibiting rate (%)=[(weight of drug-coated right ear)−(weight of non-drug-coated left ear)]×100%/(weight of non-drug-coated left ear)

The inflammation inhibiting rate of each drug is shown in table 3.

TABLE 3

| | | Inhibiting | |
|---|---|---|---|
| | | rate (%) | Inhibiting |
| | | (adding | rate (%) |
| Testing | | 0.1 w/v % | (non-caffeine |
| No. | Testing drug | of drug) | added) |
| (33) | Diphenhydramine | 20 | 20 |
| (34) | hydrochloride | 100 | 100 |
| (35) | Citric acid | 98 | 97 |
| (36) | Lactic acid | 100 | 96 |
| (37) | Malic acid | 99 | 97 |
| (38) | Tartaric acid | 100 | 98 |
| | Fumaric acid | | |

Table 3 shows that the anti-inflammation rates of traditional anti-histamine drugs are very poor in comparison with this invention (both with and without caffeine).

### Example 39 Testing in Sea Food Eating

An adult man who is very serious allergic to sea foods, especially shrimps, he ingests two capsules of this invention drug (500 mg, 125 mg of garlic, 5 mg of caffeine and 370 wt/% of citric acid) before eating shrimps. After ate many shrimps there is not any symptom of allergy at all.

### Example 40-45 Treating for Lower Limit Dosage

The oral dose of this invention such as tablet and capsule can increase the number of tablet or capsule. In case of effective component blended with food, the dosage is depending on the total amount of food eaten. The following examples will explain how the effective component of dosage is required in a food of 100 ml volume.

There are six different doses (10 mg, 60 mg, 100 mg, 200 mg, 300 mg, and 600 mg of malic acid) of testing solution, which content the basic compounds of water 100 ml, 0.1 g of propylene glycol alginate, 10 g of fructose, 300 mg of garlic, 100 mg of zinger, 10 mg of angelica ainensis radix, 3 g of honey, 10 mg of almond. These six drugs are given six groups, 5 patients per group, of catching cold patients individually per tow hours a time. The ingestion of drug is stopped when the cold syndromes are improved. The effect of drugs by treating time is listed in Table 4.

TABLE 4
Time and dose for treating cold

| Example | 40 | 41 | 42 | 43 | 44 | 45 |
|---|---|---|---|---|---|---|
| Dose, mg | 10 | 60 | 100 | 200 | 300 | 600 |
| Rate of malic acid in food, % | 0.01 | 0.06 | 0.10 | 0.2 | 0.3 | 0.6 |
| Drug containing 5% of caffeine | 5 | 2.4 | 1.5 | 1.0 | 0.7 | 0.4 |
| Time for treatment *, day | | | | | | |
| Drug without caffeine | 8 | 4.5 | 3.2 | 2.2 | 1.6 | 1.1 |
| Time for treatment *, day | | | | | | |
| ranking | Poor | good | better | best | excellent | excellent |

* the time for treatment needed is the average of the same group.

### Example 46 Injection

To dissolve 36 g of citric acid and 34 g of potassium dihydrogen citrate and 2 g of caffeine in a total volume of 1000 ml of sterilized water, then the solution is sucking filtrated through a ceramic filter, and filled in 10 ml ample by normal GMP procedure in a clean room.

### Example 47 Capsule

Grinding and compounding 350 g of citric acid, 200 g of garlic, 50 g of zinger, 10 g of tang-kuei, 10 g of almond, and 300 g of fructose, and the compound is encapsulated to 1000 pieces of product.

### Example 48 Granular and Tablet

The formulation comprises 30 g of maleic acid, 20 g of corn starch, 20 g of lactose, 5 g of Ca-CMC, 5 g of polyethylene pyrrolidone, 10 g of talc, and 6 g of caffeine. To grind maleic acid, corn starch and lactose to fine powder, then the compound is produced in a product of 1˜2 m/m granular by normal granular machine, using 5% water solution of poly ethylene pyrrolidone as a binder.

To mix talc and the produced granular, and then product of 100 tablets of containing 300 mg maleic acid are produced by tablet machine.

### Example 49 Powder

The formulation comprises 50 g of fumaric acid, 400 g of microcrystalline cellulose and 550 g of corn starch and 3 g of caffeine. To dissolve the fumaric acid with 200 ml of pure water and being adsorbed by microcrystalline cellulose, the product is dried and then mixed with corn starch to form a twenty folds powder.

### Example 50 Coffee

### Instant Coffee and Packed Coffee Solution

The formulation comprises 10 kg of coffee bean, 1.5 kg of malic acid, 9.6 kg of sugar, 7.2 kg of cream, and water for balance.

Coffee beans are roasted, ground and heat water extracted under pressure, and a 30% coffee of 10 l solution is obtained. The malic acid is added into the resulted solution. The solution is concentrated by the frozen method and frozen dried under nitrogen gas. A 4.5 kg of instant coffee product containing 33% of malic acid is produced.

That coffee product is further compounded with 9.6 kg of sugar and 7.2 kg of cream, and packed in a 17 g content product of carry-pack instant coffee.

A kind of liquid coffee drinks are made from the 30% coffee contained solution. That is compounding with 1.5 kg of malic acid, 9.6 kg of sugar, 7.2 kg of cream and the balance of water to make up of 240 liters. After heating and cooling, to pack in a volume of 200 ml, then 1200 packs of liquid coffee are produced.

### Example 51-55 Tincture and Treatment for Inflammation, Analgesic and Itchy

The formulation comprises 10 g of citric acid, 5 g of glycerin, 90 ml of alcohol (70 v/v), and 500 mg of caffeine in a mixture.

A series of testing are carried out by a group of five patients for each syndromes, which treating the topical disease three times a day, the results is shown as table 6.

TABLE 6
Results of treating inflammation, analgesic and itchy

| Example | Diseases | Treating results (*) |
|---|---|---|
| 51 | Acne(ache) | 16 h (1 day) scaled, pain improved |
| 52 | Insect bite (itchy, | 15 minutest (30 minutes Itchy |
| | inflammation, ache) | disappeared in half hour, |
| | | inflammation disappeared after |
| | | 1 h (3 h), and pain improved |
| 53 | Pruigo(itchy) | 10 h(1 day) improved |
| 54 | Skin wound(ache) | After dried the wound released, |
| | | pain improved, healing quickly |
| 55 | Pustules (ache) | 13 h (1 day)The pustules shrunk |
| | | one day, 1 day (2 day) scaled, |
| | | pain improved |

(*) treating time needed for drugs without containing caffeine

### Example 56 Glucose Injection

### Containing Other Active Agent

To dissolve 500 g of glucose, 10 g of citric acid and 0.01 g of caffeine in 10 l of high pressure sterilized water in a clean room. The solution is filtered by ceramic filter and packing into a 500 ml injection product by the GMP method.

### Example 57-61 Testing for Ache

Analgesics containing 300 mg of malic acid, 300 mg of tartaric acid, 300 mg of citric acid, 50 mg of caffeine and 10 mg of catechin are given to 5 people who are bothered by headache or physiological ache and after ingestion examining the effect with time. The results are shown in Table 6.

TABLE 6
Time needed for alleviation

| Example | Kind of ache | Time for the alleviation (minute) |
|---|---|---|
| 57 | Headache | 10 |
| 58 | Headache | 13 |
| 59 | Headache | 16 |
| 60 | Physiological ache | 26 |
| 61 | Physiological ache | 30 |

### Example 62-66 Testing for Analgesic

The components of caffeine of example 62-66 are removed, and carried out the same testing. It is obviously that the drugs without caffeine are needed more time to alleviate the pain, as shown in Table 7.

TABLE 7
Time needed for alleviation

| Example | Kind of pain | Time for the alleviation (minute) |
|---|---|---|
| 62 | Headache | 30 |
| 63 | Headache | 35 |
| 64 | Headache | 49 |
| 65 | Physiological ache | 65 |
| 66 | Physiological ache | 70 |

### Example 67 Testing for Non-Addiction Coffee

To give coffee drinks containing 300 mg of malic acid and 200 mg of tartaric acid to five people at 5 pm, whose sleeping are affected by coffee drinking. They had had good sleeping at night even although not very soundness; they had neither yawn and stretch nor suffering insomnia next morning. That is caused by the effect of detoxication of caffeine. Thus, coffee drinking will be more popularity.

### Example 68 Abstin from Caffeine Addiction

To five heavy caffeine addicted people with drugs in addition to the components contained as used in example 50, 100 mg of garlic and 1 mg of vitamin B2 are compounded and treated one week. The syndromes are improved.

### Example 69 Cholesterol Treatment

Five cardiovascular disease patients, age from 50 to 65 old, their serum cholesterol content were determined before treatment. They all have serum cholesterol higher than 200 mg/dL and the triglyceride greater than 180 200 mg/dL. Their serum cholesterol and triglyceride values are 200 mg/dL and 180 mg/dL respectively. Three pieces capsule of drug (150 mg of glyconolactone, 200 mg of citric acid, 150 mg of garlic and 10 mg of caffeine) were administrated before meals and sleeping every day for four weeks. After 12 hours fasting, they were subject to blood test for the cholesterol, triglyceride high density cholesterol and low density cholesterol, and the results are shown as Table 8.

TABLE 8
Blood test results

| | Before | After | |
|---|---|---|---|
| | treatment | treatment | |
| | Average | Average | Inhibiting |
| Items | value of 5 | value of 5 | rate % |
| Total cholesterol | 204.9 | 180.4 | −12.0 |
| Triglyceride | 210.5 | 160.1 | −23.9 |
| High density | 41.6 | 43.8 | +5.3 |
| cholesterol (HDL-C) | | | |
| Low density | 160.7 | 136.5 | −15.0 |
| cholesterol (HDL-C) | | | |
| Platelet | 230 | 141 | −30.1 |
| (10³/μL) | | | |

As shown in Table 8, the total cholesterol decreases 12.0%; triglyceride decreases 23.9%; HDL-C increases 5.3%; LDL-C decreases 15.0%. The decrease of LDL-C induces the decrease of total cholesterol in plasma, and causes the decrease of Cardiovascular risk ratio that means the ratio of (total cholesterol/HDL-C) to a value of 16.7%. It is obviously that the drugs of present invention can decrease total cholesterol, triglyceride, LDL-C and cardiovascular risk ratio, by which the products of this invention are effective to prevent diseases of arteriosclerotic thrombus and inhibit cardiovascular disease.

The platelet value of blood decreases to a very low as shown in Table 8, the normal health person has platelet value of 130-400 (103/μL), that means the drugs of this invention can decrease platelet of plasma, and then decrease the chance of thrombus formation, preventing cause of apoplexy.

### Example 70 Anti-Tumors

Eight colon cancer patients, 56 to 73 year-old, were divided into two group and treated with drugs capsule contain 500 mg of malic acid, 300 mg of garlic and 5 mg of caffeine, and drugs only containing 500 mg of malic, respectively, administrating 4 capsules after meals and before sleeping. These treatments were continue and examining the disease every month until they are recovery.

The clinic results show that the time for treatment is depending on factors of the individual bodily constitution and condition of patient. The average value is six months for patients ingested drugs containing acids, garlic and caffeine; and about eight months needed for patients they ingested drugs containing acids only as shown in Table 9. It shown that the activity of cancer disease of present invention is better than traditional one.

TABLE 9
Time for treatment

| | Time for treatment (month) | | | | | | | | | |
|---|---|---|---|---|---|---|---|---|---|---|
| | 1 | 2 | 3 | 4 | 5 | 6 | 7 | 8 | 9 | 10 |
| Number of patient | | | | | | 1 | | 2 | | 1 |
| treated with | | | | | | | | | | |
| pure acids | | | | | | | | | | |
| Number of patient | | | | | 1 | 2 | 1 | | | |
| treated with pure | | | | | | | | | | |
| acids and garlic | | | | | | | | | | |
| and caffeine | | | | | | | | | | |

### Example 71 Feedstuff

A feedstuff is composed 64.5% of corn, 32.0% of soybean, 2.0% of bone meal, 1.0% of lime stone, 0.3% of citric acid, 0.05% of propionic acid, and 0.15% of tartaric acid. The components of lime stone, citric acid and tartaric acid are pulverized and mixed at first, then compounding with other components to form product.

### Example 72 Treatment of Bee Venom

Bee and snake have venoms which could cause a life and death crisis when stung by them. For the purpose of improving the effective activity of drugs of present invention, a person who ingests three pieces of drug (containing 450 mg of succinic acid, 150 mg of garlic), and thirty minutes after his arm was stung by bee three times. The treatment was first treating with tincture solution composed 10% of α-hydroxy octanoic acid, 2% of oxalacetic acid, 1% of aconitic acid, 1% of ketoglutaric acid and 2% of acetic acid; and then ingested 3 pieces of drugs every 2 hours for half day. It was all right that there was not any special inflammation or pains.

### Example 73 Analgesia and Suppuration for Tooth

It is not a best method in treating a toothache caused by a crowned tooth swelling that the ill tooth must be extracted or the crown must be destructed first. Instead of by the traditional method, two such patients were treated with ingesting 2 g of malic acid every two hours, shortly afterward their pains were killed but could not chewing. About 8 and 10 days were needed to suppurate and recovery completely when they could chew again. This method can keep the ill tooth without extraction.

### Example 74 Treatment of HIV Disease

A HIV patient whose blood testing results shown the concentrations of CD4+ T cells and virus were 129/μl and 70,000/cc respectively. After 4 weeks of treatment with ingesting three gelatin capsules of drug, composed 500 mg of malic acid, 200 mg of garlic and 5 mg of caffeine, each three hours, and testing his blood again. The results shown good antivirus effect that the concentrations of CD4+ T cells and virus were 700/μl and 0/cc respectively.

### Example 75 Treatment of Influenza

A 69 years old man infected with influenza shown in flu symptoms of dry cough, cold and fever, fatigue, loss of appetite and headache. Treating with drug composing 200 mg of malic acid, 100 mg of succinic acid, 100 mg of citric acid, 100 mg of tartaric acid, 170 mg of garlic, 30 mg of ginger and 10 mg of caffeine, ingested three pieces of this every two hours. One day after the symptoms were much improved, and recovery almost at second day.

### Example 76 Preventing Food Allergic Health Care Fish Cans

10 kg of sardines are washed. After their heads and tails being cut and the inner organics being cleaned up, they are cut into a proper size. These raw materials are cooked in a 20 L solution that contains 1.2 kg of salt, 20 mg of caffeine and 800 kg of citric acid. The cooked fish then is canned into No. 4 size steel can with 75 g of tomato ketchup, and the product then is sterilized by normal process.

### Example 77 Preventing Food Allergic Health Care Cookies

The formulation comprises 10 kg of wheat powder, 3.5 kg of sugar, 0.8 kg of shortening oil, 1 kg of millet jelly, 0.03 kg of salt, 0.2 kg of ferment, and 0.62 kg of α-hydroxy ethanoic acid. To follow the traditional method of cake making, the solids of wheat powder, sugar, salt, and α-hydroxy ethanoic acid are ground and sieved individually first. They are mixed them with ferment and part of wheat powder, and compounded well with millet jelly, caffeine and shortening oil. After shaping, and baking in two stages; first stage is at 180˜200° C., and the second stages is at 150˜205° C.; products are produced.

### Example 78 Preventing Food Allergic Health Care Cakes

The formulation comprises 1 kg of wheat powder, 1 kg of sugar, 1 kg of egg, 150 g of gluconolactone, 2 g of caffeine and 300 g of water. The albumin and egg-yellow are separated first, and the former is bubbled by bating. After the albumin is bubbled, sugar, gluconolactone, caffeine and water are added and mixed homogeneously. The wheat powder is sieved and added to the mixture. To mix quietly and being molded for baking, then cakes are produced.

### Example 79 Preventing Food Allergic Health Care Candies

The formulation comprises 430 g of white sugar, 350 g of starch syrup, 170 g of inverted syrup, 50 g of gelatin, 20 g of potassium dihydrogen citrate, 1 g of caffeine, 20 g of sodium dihydrogen citrate, and 2 ml of vanilla extract. Gelatin is cut into pieces before dissolving in triple volumes of water, and heated with steam in a doubled layer bottom kettle. Following the process of soft candy making method; dissolving sugar, starch syrup and inverted syrup; cooking; adding potassium dihydrogen citrate, sodium dihydrogen citrate, caffeine and vanilla extract; mixing even; adding dissolved gelatin; mixing carefully; degassing; powdering molding; cutting; packing; and finally the products are obtained.

### Example 80 Preventing Food Allergic Health Care Mineral Containing Lactic Acid Drinks

The formulation comprises 1 kg of skim milk, 1.5 kg of sugar, 15 g of lactic acid, 5 g of calcium lactate, 4 g of propylene glycol alginate and 200 mg of caffeine. Skim milk is heated to 50° C. when sugar is dissolved. Then calcium lactate, caffeine and propylene glycol alginate are added and to keep at 80° C. for 20 minutes. After sterilizing, the solution is filtered and cooled down to 15° C. The lactic acid is mixed with 75 ml of boiled water and added to the filtered skim milk solution in stirring, and finally bottled to obtain product.

### Example 81 Preventing Food Allergic Health Care Peanut Products

The formulation comprises 1 kg of peanut, 20 g of salt, 25 g of fumaric acid, 50 g of lecithin, 20 mg of pineapple enzyme, 2 ml of ethanol and 300 mg of caffeine. The peanut is roasted at 160° C. for 1 hour and ground into powder after drying, and sieved to remove the skins and germs. To add salt, lecithin, pineapple enzyme (which is dissolved in alcohol first), caffeine and fumaric acid consequently, and is ground to form paste before packing in a 500 g bottle.

### Example 82 Preventing Food Allergic Health Care Puddings

The formulation comprises 750 ml of milk, 6 pieces of egg, 150 g of sugar, 21 g of succinic acid, 2 drops of ethyl iso-valerianate, 150 mg of caffeine and caramel raw material (100 g of sugar and 6 g of water) for 10 pieces puddings. The process is: making caramel by heating the mixture of sugar and water in flat pan; the caramel is divided into 10 portions for vessels which the bottoms have rubbed with few amount of oil; heating the mixture of milk, caffeine and perfume to near boiling by steam; mixture of egg and sugar is bubbled and added to the milk mixture; mixing the resulted mixture; then is filled into the vessels carefully; and steamed at 160° C. for 30 minutes to form the product.

### Example 83 Preventing Food Allergic Health Care Orange Juice Drinks

The formulation comprises 5 kg of orange juice (sweetness 10° and acidity 1.0%), 0.95 kg of anhydrous fructose, 1 ml of orange essence, 5 g of caffeine and 150 g of citric acid. The production method is to mix the dissolved materials, and pure water is added to make up 10 l of orange juice and packed.

### Example 84 Preventing Food Allergic Health Care Food

The formulation comprises 100 g of malic acid, 100 g of tartaric acid, 100 g of α-hydroxy octanoic acid, 200 g of yam, 100 g of garlic, 10 g of caffeine and 6 g of carroten. The materials are compounded and filled into 1000 capsules. People who are allergic to shrimps food can be improved by ingesting two capsules of this drug in advance to eat. All the preventing food allergic health care food described previously are applied to all people who are hypersensitivity to food can be improved.

### Example 85 Tests for Increase the Ability of Learning and Memory

Testing were carried out for increasing the ability of learning and memory by ingesting drug, each piece containing 600 mg, selected from group comprising fumaric acid, maleic acid, succinic acid, malic acid, tartaric acid, citric acid, lactic acid, α-hydroxy octanoic acid, gluconolacton, α-hydroxy ethanoic acid, keto-glutaric acid and aconitic acid. A 68 year-old man administrates see a random series of 100 numbers, typed by PC, for 10 minutes and memory it. Then recite and write down the memorized number series. To check the recited series with the original one to see how many numbers are correct. In each testing at least has 10 minutes interval, and repeat four times. Then, 30 minutes after, ingesting three pieces of one of the drugs and repeating the process as described four times. Each kind of drugs testing is done in half day, morning or afternoon only.

The testing results were counted by one correct number for one point. Make the average value of four times testing of before and after drug ingesting, respectively. The increase of learning or memory ability is calculated by following equation and listed in Table 14:

Rate of increase of memory ability=[(average value of four time testing before ingesting drug)−(average value of four times testing after ingesting drug)]×100%/(average value of four time testing before ingesting drug)

TABLE 14
Rate of increase memory ability

| Drug | Increase rate (%) |
|---|---|
| Fumaric acid | 300 |
| Malic acid | 315 |
| Lactic acid | 280 |
| α-hydroxy ethanoic acid | 300 |
| Oxalacetic acid | 310 |
| Maleic acid | 370 |
| Tartaric acid | 305 |
| α-hydroxy octanoic acid | 300 |
| keto-glutaric acid | 321 |
| Acetic acid | 307 |
| Succinic acid | 330 |
| Citric acid | 360 |
| gluconolacton | 290 |
| aconitic acid. | 315 |
| Propionic acid | 296 |

The testing results show that the drugs of this invention have remarkable effect in increasing the abilities of learning and memory as shown in Table 14.

1-23. (canceled)

24: A pharmaceutical composition for lowering humoral pH, said pharmaceutical composition comprising:
0.05-99.9 wt % of edible carboxylic acid selected from the group consisting of fumaric acid, succinic acid, α-hydroxyl acids, malic acid, tartaric acid, citric acid, lactic acid, oxalactic acid, α-hydroxy octanoic acid, gluconolactone, glycolic acid, acetic acid, and their acidic salts of sodium and potassium;
0.1-6 wt % of caffeine;
0-80 wt % of at least one herb; and
0-96 wt % of acceptable carrier;
wherein the amount of said edible carboxylic acid is greater than that of caffeine.

25: The pharmaceutical composition as set forth in claim 24, said caffeine is selected from the group consisting of caffeine, coffee extracts, coffee powder, and extracts of caffeine containing materials.

26: The pharmaceutical composition as set forth in claim 25, wherein said herb is selected from the group consisting of Gambier, areca and its products, garlic, leek, chive, shallot, ramson, scallion, zinger, tang-kuei, licorice, astragali radix, almonds, ginseng, atractylodis rhizoma, pinelliae tuber, angelica sinensis radix, hoelen, asini gelatinum, dried tangerine, asparagi radix, rehmanniae radix et rhizoma, perillae fructus, perillae caulis, anemarrhenae rhizoma, albae sinapis semen, mori radicis cortex, zingiberis siccatulm rhizoama, yam, carroten, lily bulbous, sesame, ginseng powder, ginseng extracts, processed fruits, minerals, vitamins, powder milk, peanut products, vegetable seed oil, cooked foods, and amino acids.

27: The pharmaceutical composition as set forth in claim 26, wherein said acceptable carrier is a pharmaceutical acceptable carrier selected from the group consisting of binders, thickeners, softeners, dispersions, emulsifiers, preservatives, lubricants, enzymes, sweeteners, perfumers, and pigments.

28: The pharmaceutical composition as set forth in claim 27, wherein the amount of said edible carboxylic acid is at least three times greater than that of caffeine.

29: The pharmaceutical composition as set forth in claim 28, wherein said edible carboxylic acid is 0.1-10 wt %, said caffeine is 1-3 wt %.

30: The pharmaceutical composition as set forth in claim 27, wherein said edible carboxylic acid is processed fruit containing said edible carboxylic acid, and wherein said pharmaceutical composition is in the form selected from the group consisting of oral food, beverage, candy, biscuits, capsule, tablet, flake, granular, powder, pile, syrup, solution, and suspension.

31: The pharmaceutical composition as set forth in claim 27, wherein said herb is garlic for treating cancer.

32: The pharmaceutical composition as set forth in claim 24, wherein said edible carboxylic acid is 14-89.9 wt %, and said herb is 10-80 wt % of garlic.

33: The pharmaceutical composition as set forth in claim 24, wherein said edible carboxylic acid is 0.05-5 wt % and said acceptable carrier is 0.05-10 wt % of a feedstuff acceptable carrier, and further comprising 85-99.9 wt % of feedstuff.

34: The pharmaceutical composition as set forth in claim 27, wherein said edible carboxylic acid is 15-85%, said caffeine is 1-3%, and wherein said acceptable carrier is a drink acceptable carrier, and the amount of said caffeine in each dose is less than 200 mg.

35: The pharmaceutical composition as set forth in claim 27, wherein said edible carboxylic acid is 15-85 wt %, said caffeine is 1-3 wt %.

36: The pharmaceutical composition as set forth in claim 27, wherein said pharmaceutical composition is used to prevent, treat or alleviate conditions selected from the group consisting of allergy diseases, ache, infection, cold, thrombus or clotting coagulation during transfusion or dialysis, inflammation, cancer, virus infection, intoxication, memory decay, and caffeine addiction.

37: A pharmaceutical composition for lowering humoral pH, said pharmaceutical composition comprising:
0.05-99.9 wt % of edible carboxylic acid selected from the group consisting of fumaric acid, succinic acid, α-hydroxyl acids, malic acid, tartaric acid, citric acid, lactic acid, oxalactic acid, α-hydroxy octanoic acid, gluconolactone, glycolic acid, acetic acid, and their acidic salts of sodium and potassium;
0.1-6 wt % of caffeine selected from the group consisting of caffeine, coffee extracts, coffee powder, and extracts of caffeine containing materials;
0-80 wt % of at least one herb selected from the group consisting of Gambier, areca and its products, garlic, leek, chive, shallot, ramson, scallion, zinger, tang-kuei, licorice, astragali radix, almonds, ginseng, atractylodis rhizoma, pinelliae tuber, angelica sinensis radix, hoelen, asini gelatinum, dried tangerine, asparagi radix, rehmanniae radix et rhizoma, perillae fructus, perillae caulis, anemarrhenae rhizoma, albae sinapis semen, mori radicis cortex, zingiberis siccatulm rhizoama, yam, carroten, lily bulbous, sesame, ginseng powder, ginseng extracts, processed fruits, minerals, vitamins, powder milk, peanut products, vegetable seed oil, cooked foods, and amino acids; and
0-96 wt % of acceptable carrier selected from the group consisting of binders, thickeners, softeners, dispersions, emulsifiers, preservatives, lubricants, enzymes, sweeteners, perfumers, and pigments;
wherein the amount of said edible carboxylic acid is greater than that of caffeine.

38: A method of preventing, treating, or alleviating conditions by lowering humoral pH, which comprises the step of administering a pharmaceutical composition comprising:
0.05-99.9 wt % of edible carboxylic acid selected from the group consisting of fumaric acid, succinic acid, α-hydroxyl acids, malic acid, tartaric acid, citric acid, lactic acid, oxalactic acid, α-hydroxy octanoic acid, gluconolactone, glycolic acid, acetic acid, and their acidic salts of sodium and potassium;
0.1-6 wt % of caffeine;
0-80 wt % of at least one herb; and
0-96 wt % of acceptable carrier;
wherein the amount of said edible carboxylic acid is greater than that of caffeine.

39: The method as set forth in claim 38, wherein said caffeine is selected from the group consisting of caffeine, coffee extracts, coffee powder, and extracts of caffeine containing materials.

40: The method as set forth in claim 39, wherein said herb is selected from the group consisting of Gambier, areca and its products, garlic, leek, chive, shallot, ramson, scallion, zinger, tang-kuei, licorice, astragali radix, almonds, ginseng, atractylodis rhizoma, pinelliae tuber, angelica sinensis radix, hoelen, asini gelatinum, dried tangerine, asparagi radix, rehmanniae radix et rhizoma, perillae fructus, perillae caulis, anemarrhenae rhizoma, albae sinapis semen, mori radicis cortex, zingiberis siccatulm rhizoama, yam, carroten, lily bulbous, sesame, ginseng powder, ginseng extracts, processed fruits, minerals, vitamins, powder milk, peanut products, vegetable seed oil, cooked foods, and amino acids.

41: The method as set forth in claim 40, wherein said acceptable carrier is a pharmaceutical acceptable carrier selected from the group consisting of binders, thickeners, softeners, dispersions, emulsifiers, preservatives, lubricants, enzymes, sweeteners, perfumers, and pigments.

42: The method as set forth in claim 41, wherein said conditions are selected from the group consisting of allergy diseases, ache, infection, cold, thrombus or clotting coagulation during transfusion or dialysis, inflammation, cancer, virus infection, intoxication, memory decay, caffeine addiction, and side effects of the drug Paclitaxel.

43: A pharmaceutical composition for lowering humoral pH, said pharmaceutical composition comprising:
0.05-100 wt % of edible carboxylic acid selected from the group consisting of fumaric acid, α-hydroxyl acids, malic acid, tartaric acid, citric acid, lactic acid, oxalactic acid, α-hydroxy octanoic acid, gluconolactone, glycolic acid, acetic acid, and their acidic salts of sodium and potassium; in which acetic acid used in treatments of both cold and inflammation are excluded; fumaric acid and its acidic salts used in treatments of transplantation, toxication and psoriasis are excluded;
0-80 wt % of at least one herb; and
0-96 wt % of acceptable carrier.

44: The pharmaceutical composition as set forth in claim 43, wherein said herb is selected from the group consisting of Gambier, areca and its products, garlic, leek, chive, shallot, ramson, scallion, zinger, tang-kuei, licorice, astragali radix, almonds, ginseng, atractylodis rhizoma, pinelliae tuber, angelica sinensis radix, hoelen, asini gelatinum, dried tangerine, asparagi radix, rehmanniae radix et rhizoma, perillae fructus, perillae caulis, anemarrhenae rhizoma, albae sinapis semen, mori radicis cortex, zingiberis siccatulm rhizoama, yam, carroten, lily bulbous, sesame, ginseng powder, ginseng extracts, processed fruits, minerals, vitamins, powder milk, peanut products, vegetable seed oil, cooked foods, and amino acids.

45: The pharmaceutical composition as set forth in claim 43, wherein said acceptable carrier is a pharmaceutical acceptable carrier selected from the group consisting of binders, thickeners, softeners, dispersions, emulsifiers, preservatives, lubricants, enzymes, sweeteners, perfumers, and pigments.

46: The pharmaceutical composition as set forth in claim 45, wherein said edible carboxylic acid is 0.1-10 wt %.

47: The pharmaceutical composition as set forth in claim 45, wherein said edible carboxylic acid is processed fruit containing said edible carboxylic acid, and wherein said pharmaceutical composition is in the form selected from the group consisting of oral food, beverage, candy, biscuits, capsule, tablet, flake, granular, powder, pile, syrup, solution, and suspension.

48: The pharmaceutical composition as set forth in claim 43, wherein said edible carboxylic acid is 14-89.9 wt %, and said herb is 10-80 wt % of garlic.

49: The pharmaceutical composition as set forth in claim 43, wherein said edible carboxylic acid is 0.05-5 wt % and said acceptable carrier is 0.05-10 wt % of a feedstuff acceptable carrier, and further comprising 85-99.9 wt % of feedstuff.

50: The pharmaceutical composition as set forth in claim 45, wherein said edible carboxylic acid is 15-85%, and wherein said acceptable carrier is a drink acceptable carrier.

51: The pharmaceutical composition as set forth in claim 45, wherein said pharmaceutical composition is used to prevent, treat or alleviate conditions selected from the group consisting of allergy diseases, ache, infection, cold, inflammation, virus infection, and caffeine addiction.

52: A pharmaceutical composition for lowering humoral pH, said pharmaceutical composition comprising:
0.05-100 wt % of edible carboxylic acid selected from the group consisting of fumaric acid, α-hydroxyl acids, malic acid, tartaric acid, citric acid, lactic acid, oxalactic acid, α-hydroxy octanoic acid, gluconolactone, glycolic acid, acetic acid, and their acidic salts of sodium and potassium; in which acetic acid used in treatments of both cold and inflammation are excluded; fumaric acid and its acidic salts used in treatments of transplantation, toxication and psoriasis are excluded,
0-80 wt % of at least one herb selected from the group consisting of Gambier, areca and its products, garlic, leek, chive, shallot, ramson, scallion, zinger, tang-kuei, licorice, astragali radix, almonds, ginseng, atractylodis rhizoma, pinelliae tuber, angelica sinensis radix, hoelen, asini gelatinum, dried tangerine, asparagi radix, rehmanniae radix et rhizoma, perillae fructus, perillae caulis, anemarrhenae rhizoma, albae sinapis semen, mori radicis cortex, zingiberis siccatulm rhizoama, yam, carroten, lily bulbous, sesame, ginseng powder, ginseng extracts, processed fruits, minerals, vitamins, powder milk, peanut products, vegetable seed oil, cooked foods, and amino acids; and
0-96 wt % of acceptable carrier selected from the group consisting of binders, thickeners, softeners, dispersions, emulsifiers, preservatives, lubricants, enzymes, sweeteners, perfumers, and pigments.

53: A method of preventing, treating, or alleviating conditions by lowering humoral pH, which comprises the step of administering a pharmaceutical composition comprising:
0.05-100 wt % of edible carboxylic acid selected from the group consisting of fumaric acid, α-hydroxyl acids, malic acid, tartaric acid, citric acid, lactic acid, oxalactic acid, α-hydroxy octanoic acid, gluconolactone, glycolic acid, acetic acid, and their acidic salts of sodium and potassium; in which acetic acid used in treatments of both cold and inflammation are excluded; fumaric acid and its acidic salts used in treatments of transplantation, toxication and psoriasis are excluded;
0-80 wt % of at least one herb; and
0-96 wt % of acceptable carrier.

54: The method as set forth in claim 53, wherein said herb is selected from the group consisting of Gambier, areca and its products, garlic, leek, chive, shallot, ramson, scallion, zinger, tang-kuei, licorice, astragali radix, almonds, ginseng, atractylodis rhizoma, pinelliae tuber, angelica sinensis radix, hoelen, asini gelatinum, dried tangerine, asparagi radix, rehmanniae radix et rhizoma, perillae fructus, perillae caulis, anemarrhenae rhizoma, albae sinapis semen, mori radicis cortex, zingiberis siccatulm rhizoama, yam, carroten, lily bulbous, sesame, ginseng powder, ginseng extracts, processed fruits, minerals, vitamins, powder milk, peanut products, vegetable seed oil, cooked foods, and amino acids.

55: The method as set forth in claim 53, wherein said acceptable carrier is a pharmaceutical acceptable carrier selected from the group consisting of binders, thickeners, softeners, dispersions, emulsifiers, preservatives, lubricants, enzymes, sweeteners, perfumers, and pigments.

56: The method as set forth in claim 53, wherein said conditions are selected from the group consisting of allergy diseases, ache, infection, cold, inflammation, virus infection, caffeine addiction and side effects of the drug Paclitaxel.
